(19) United States
(12) Patent Application Publication
(10) Pub. No.: US 2005/0170023 A1
(43) Pub. Date: Aug. 4, 2005

(54) Composition, process for making using pressurized fluid, and uses thereof

(75) Inventor: Roland De La Mettrie, Le Vesinet (FR)

Correspondence Address: OBLON, SPIVAK, MCCLELLAND, MAIER & NEUSTADT, P.C., 1940 DUKE STREET, ALEXANDRIA, VA, 22314, US

(73) Assignee: L'OREAL, Paris (FR)

(21) Appl. No.: 11/037,376

(22) Filed: Jan. 19, 2005

Related U.S. Application Data
(60) Provisional application No. 60/545,189, filed on Feb. 18, 2004.

(30) Foreign Application Priority Data
Jan. 29, 2004 (FR) 0400846

(51) Int. Cl. A61K 35/78; A61K 38/08; A61K 38/06

(52) U.S. Cl. 424/750; 514/16; 514/17; 514/18; 424/757; 424/769

(57) ABSTRACT

A process for preparing a composition by percolating a fluid containing steam, at a pressure of at least 3 bar, through at least one member selected from an amino acid, oligopeptide, peptide, optionally modified, optionally hydrolysed protein, and mixtures thereof, in solid or pasty form.

### REFERENCE TO PRIOR APPLICATIONS

This application claims priority to U.S. provisional application 60/545,189 filed Feb. 18, 2004, and to French patent application 0400846 filed Jan. 29, 2004, both incorporated herein by reference.

### FIELD OF THE INVENTION

The present invention relates to a process for preparing a composition useful for example for the treatment of keratin materials, in particular the skin and human keratin fibres such as the hair, to the composition prepared, and also to a cosmetic process for treating keratin materials using this composition.

Additional advantages and other features of the present invention will be set forth in part in the description that follows and in part will become apparent to those having ordinary skill in the art upon examination of the following or may be learned from the practice of the present invention. The advantages of the present invention may be realized and obtained as particularly pointed out in the appended claims. As will be realized, the present invention is capable of other and different embodiments, and its several details are capable of modifications in various obvious respects, all without departing from the present invention. The description is to be regarded as illustrative in nature, and not as restrictive.

### BACKGROUND OF THE INVENTION

In cosmetics, it is always sought to improve the cosmetic properties of keratin materials, for example sensitized hair, i.e. hair that has been damaged or embrittled by the chemical action of atmospheric agents such as free radicals, light and pollution, and/or hair treatments such as permanent-waving, dyeing or bleaching. (cosmetic treatment) compositions comprising conditioning agents such as amino acids, oligopeptides, peptides, and optionally modified, optionally hydrolysed proteins, intended to repair or limit the harmful or adverse effects produced by the various treatments or attacking factors to which keratin materials are more or less repeatedly subjected, may thus be applied to these materials. These conditioning agents may also improve the cosmetic properties of natural hair.

Agents for conditioning keratin materials, such as amino acids, oligopeptides, peptides, and optionally modified, optionally hydrolysed proteins, in particular protect wet or dry hair against external attack, reinforce the hair fibres and give the hair body.

These conditioning agents also give the skin anti-ageing care and allow protection of the collagen, and also cell nutrition.

However, (cosmetic treatment) compositions containing such conditioning agents are generally aqueous compositions in which the agents must be dissolved. The lack of solubility of these compounds reduces the conditioning power of these compositions. In addition, this solubility criterion reduces the number of amino acids, oligopeptides, peptides, and optionally modified, optionally hydrolysed proteins that can be used for the cosmetic treatment of keratin materials. This is particularly the case for compounds with a high melting point.

### DETAILED DESCRIPTION OF THE PREFERRED EMBODIMENTS

It has now been discovered, surprisingly, that by using a novel process for preparing a composition, compositions that are more or less concentrated in conditioning agent(s) may be obtained in a very short time, for example of less than 2 minutes, according to need, especially without preserving agent, enabling the solubility problems outlined above to be overcome.

This process may be performed simply and is suited to the needs of the consumer. A fluid, the temperature of which is preferably greater than or equal to 30° C., is passed, under pressure, for a very short time, for example of less than 1 minute, through at least one conditioning agent in solid or pasty form, preferably solid and even more preferably pulverulent.

It also enables the use in anhydrous form of conditioning agents that are unstable in aqueous compositions either because they react with water or because they react in aqueous solution with compounds that do not react with them in an anhydrous composition.

The compositions prepared according to this process may have limited stability on storage, which is not a drawback in this case since the process leads to a ready-to-use composition intended to be used quickly after its preparation, for example within five minutes of preparation, especially after cooling to a temperature that is acceptable for keratin materials, preferably below 60° C., better still below 50° C. The composition may also be used up to one week or more after its preparation, depending on the rate of degradation of the conditioning agent used.

Given the very short preparation time, the compositions of the invention, which may in particular be (cosmetic treatment) compositions, may be prepared “on demand” by mixing different cosmetically active compounds according to the desired cosmetic properties.

According to another embodiment, it is not necessary to determine beforehand the concentrations of said agents in solution, which limits the measuring errors by the user, because the conditioning agents may be packaged in a ready-to-use device.

In addition, the process according to the invention makes it possible to avoid the use of multi-compartment bottles, which makes the process particularly economical and safer for the user.

The composition thus obtained may be used alone or as a mixture with another composition.

A further advantage of this preparation process is the production of compositions that have better cosmetic properties. In particular, keratin fibres treated with a composition obtained via the process according to the invention have improved conditioning properties, especially better protection of the hair against external attack, better reinforcement of the hair fibres and better body, and the skin treated in this way is thus better protected against ageing and more nourished.

One subject of the invention is thus a process for preparing a (cosmetic) composition useful for treating keratin materials, comprising percolating a fluid at a pressure of at least 3 bar through at least one of an amino acid, oligopeptide, peptide, and/or optionally modified, optionally hydrolysed protein, in solid or pasty form.

Another subject of the invention is a composition that may be obtained via the process according to the invention.

A subject of the invention is also the use of the composition obtained according to the process of the invention, for the cosmetic treatment of keratin materials, and especially for conditioning the hair and the skin.

Another subject of the invention is a packaging device for performing the preparation process of the present invention.

Other subjects, characteristics, aspects and advantages of the invention will emerge even more clearly on reading the further nonlimiting description and the examples that follow.

According to the invention, the process for preparing a (cosmetic) composition for treating keratin materials comprises percolating fluid, at a temperature preferably of greater than or equal to 30° C., better still ranging from 30° C. to 150° C. and even more preferably ranging from 40° C. to 120° C., at a pressure of at least 3 bar (3×105 Pa), through at least one of an amino acid, oligopeptide, peptide, and an optionally modified, optionally hydrolysed, protein, in solid or pasty form.

Percolation is a movement of fluid through a porous medium, allowing the passage of the fluid due to the action or effect of pressure.

The fluid preferably comprises at least steam. It may be steam optionally accompanied by at least one of liquid water, one or more cosmetically acceptable liquid and/or gaseous solvents, and mixtures thererof. Preferably, the fluid is steam possibly accompanied by liquid water.

Examples of organic solvents that may be used herein include C1-C4 lower alcohols, such as ethanol and isopropanol; polyols and polyol ethers, for instance 2-butoxyethanol, propylene glycol, propylene glycol monomethyl ether, diethylene glycol monomethyl ether and diethylene glycol monoethyl ether, and also aromatic alcohols, for instance benzyl alcohol or phenoxyethanol, and mixtures thereof.

The amino acids, oligopeptides, peptides, and optionally modified, optionally hydrolysed proteins that may be used in the invention are in solid or pasty form, preferably in solid form and even more preferably pulverulent.

For the purposes of the present invention, the term “pasty form” means a consistency intermediate between a solid phase and a liquid phase. The viscosity of this pasty phase is preferably greater than 0.1 Pa.s and even more preferably greater than 1 Pa.s, at 25° C. with a shear rate of 10 s-l.

The term “keratin materials” means the skin, the lips, and/or the integuments such as the nails and keratin fibres, for example the eyelashes, the eyebrows and the hair.

The process of the present invention may be performed using a standard device for generating a pressurized fluid at a temperature preferably of greater than or equal to 30° C., better still ranging from 30° C. to 150° C., and even more preferably from 40° C. to 120° C. Such a device preferably comprises a pressure-resistant chamber equipped with a thermal block, and also a circuit for conveying the fluid to the amino acid, oligopeptide, peptide, and/or optionally modified, optionally hydrolysed protein used in the invention.

According to another embodiment, the device additionally comprises a reservoir of liquid(s) and also a pump for conveying the liquid(s) to the chamber.

The liquid contained in the reservoir is preferably the one to be percolated, such as either water, or a mixture of water and of one or more cosmetically acceptable solvents. Preferably the liquid it is water.

A device that is particularly useful for performing the process of the present invention is a coffee machine of the “espresso” type. Such machines are well known in the art. For example, these machines are described in patents AT 168 405, U.S. Pat. No. 2,688,911, DE 324 33 870 and IT 1 265 636.

According to one particular embodiment of the invention, the percolation step is performed with a fluid at a temperature of greater than or equal to 30° C., better still ranging from 30° C. to 150° C., and even more preferably from 40° C. to 120° C., under a pressure ranging from 10 to 30 bar (3×105 to 3×106 Pa), preferably of at least 4 bar (4×105 Pa), more preferably greater than or equal to 10 bar (106 Pa) and most particularly ranging from 10 to 30 bar (106 to 3×106 Pa).

A cosmetic composition comprising at least one amino acid, oligopeptide, peptide, and optionally modified, optionally hydrolysed protein may be used directly in the device for generating the pressurized fluid in a container intended for this use. It may also be packaged in a particular packaging device, comprising a closed housing delimited by at least one wall that is at least partially permeable to fluid under a pressure of at least 3 bar. Such devices are described, for example, in patent applications WO 00/56629, EP 512 470, U.S. Pat. No. 5,897,899 or WO 99/03573. These packaging devices are generally protected against the air, humidity and/or light.

According to one particular embodiment, the housing is delimited by two sealed sheets. According to another embodiment, the housing is delimited by a tray closed with a lid.

These devices may be manufactured from woven or nonwoven, plastic or plant materials, for example cellulose, metal such as aluminium, or composite materials. Such devices are described, for example, in patent applications WO 00/56629, EP 512 470 or WO 99/03573.

The amino acids that may be used according to the invention may be natural or synthetic, and comprise at least one acid function. The acid function(s) may be carboxylic, sulfonic, phosphonic or phosphoric.

Included as amino acids that may be used in the present invention are aspartic acid, glutamic acid, alanine, arginine, asparagine, carnitine, cysteine, glutamine, glycine, histidine, isoleucine, leucine, methionine, N-phenylalanine, proline, serine, taurine, threonine, tryptophan, tyrosine and valine.

It is also possible to use polyamino acids such as polylysine, polyarginine and polyaspartic acid, for example.

The amino acids that are particularly preferred in the present invention are arginine, cysteine, glycine and taurine.

As examples of oligopeptides, peptides and hydrolysed or non-hydrolysed, modified or unmodified proteins used in the invention, mention may be especially of:

synthetic oligopeptides such as TYR-GLY-GLY-PHE-LEU, H-LYS-THR-THR-DAP-SER-OH triacetate, HIS-SER-GLN-GLY-THR-PHE, palmitoyl-TYR-GLY-GLY-PHE-LEU and palmitoyl-GLY-GLN-PRO-ARG;

plant proteins derived from plants in native form or present in flours.
Among the native proteins that may be mentioned are soybean proteins, oat proteins, pea proteins and in particular pea albumin, apricot or kernel proteins, wheat gliadin, Triticum aestivum wheat proteins, extensin and thaumatin.
Among the flours that may be mentioned are wheatgerm flour, oat flour, soybean flour, white lupin flour, wholewheat flour, rice flour, camellia flour, barley flour, rye flour, chestnut flour, quinoa flour and corn flour;

animal proteins produced by species of the animal kingdom, such as whey proteins, casein, silk proteins (including fibroin), transglutin, lactalbumins, lactoglobulins, lactoferrins, egg white proteins including albumin, keratins including those derived from feathers, collagen and gelatin;

protein hydrolysates obtained by chemical or enzymatic hydrolysis, such as peptides or protein hydrolysates from soybean, wheat, corn, potato and pea;

chemically modified proteins or protein hydrolysates, such as the ethyl or propyl esters of wheat peptides, α-lactalbumin palmitoyl stearoyl and quaternized protein hydrolysates.

The amino acid(s), oligopeptide(s), peptide(s) and optionally modified, optionally hydrolysed proteins(s) may be used as a mixture with one or more solid or pasty, and preferably pulverulent, adjuvants. The adjuvants may be chosen from clays, salts, anionic, nonionic, cationic or zwitterionic surfactants, natural or synthetic thickeners, optionally modified starch, glass beads, silica, Nylon, alumina, titanium dioxide, zeolites, polymethyl methacrylate (PMMA), chitosan, maltodextrin, cyclodextrin, mono- or disaccharides, for instance glucose, sucrose, sorbitol or fructose, zinc oxide, zirconium oxide, silica beads, talc, borosilicates, especially calcium borosilicate, polyethylene, polytetrafluoroethylene (PTFE), cellulose and its derivatives, superabsorbent compounds, magnesium carbonate, calcium carbonate, polyacrylamide, porous hydroxyapatite, sawdust, wrack powder, crosslinked polyvinylpyrrolidone, calcium alginate, active charcoal, and poly(vinylidene chloride/acrylonitrile) particles, especially those sold under the general name “Expancel®” by the company Akzo Nobel under the particular reference “Expancel® WE” or “Expancel DE”, and mixtures thereof.

When one or more adjuvants are present, the amino acid(s), oligopeptide(s), peptide(s), and protein(s) used in the invention is (are) preferably present in an amount ranging from 0.5% to 99% by weight, better still from 1% to 80% by weight and even more preferably from 2% to 60% by weight relative to the total weight of amino acid(s), oligopeptide(s), peptide(s) and/or protein(s), and adjuvant(s) in solid or pasty form.

When plants or plant extracts are used in the process of the present invention, they may be pretreated before the percolation step. The pretreatment can include one or more of drying, roasting, cryogrinding and freeze-drying.

The cosmetic composition for treating keratin materials obtained according to the process of the invention can contain, besides the amino acid(s), oligopeptide(s), peptide(s) and/or protein(s), and the component(s) of the fluid, i.e. water and/or cosmetically acceptable solvent(s), optionally all or some of the adjuvant(s) present in the mixture in solid or pasty form.

The invention also relates to a composition obtained via the process according to the invention, a particularly preferred composition not comprising any preserving agent.

Using the preparation process of the invention, a cosmetic composition for treating keratin materials is obtained, which may be applied directly to keratin materials, or which may be mixed with, e.g., a cosmetically acceptable medium, or alternatively at least one additive conventionally used in cosmetics may be added thereto by an operator. At least two compositions obtained via the process of the invention may also be mixed together. The cosmetic composition for treating keratin materials optionally resulting from the mixture(s) and/or addition(s) indicated above will be referred to hereinbelow as the final (cosmetic treatment) composition or final composition.

One particular embodiment of the invention is applying the composition obtained by means of a device not requiring any human intervention, and optionally equipped with a cooling means.

Another particular embodiment is ingesting the composition obtained according to the process of the invention, preferably when there is no known toxicity problem in the technique, for example when the conditioning agent is taurine.

The amount of the amino acids, oligopeptides, peptides and/or proteins present in the final (cosmetic treatment) composition is generally preferably between 0.001% and 50% by weight approximately, more preferably between 0.005% and 30% by weight, and even more preferably between 0.01% and 20% by weight relative to the total weight of the final (cosmetic treatment) composition.

When the (cosmetic treatment) composition obtained via the process of the present invention is mixed with a cosmetically acceptable medium, such a medium generally comprises water or a mixture of water and of at least one organic solvent to dissolve the compounds that would not be sufficiently soluble in water.

The term “cosmetically acceptable” means a medium that is compatible with keratin materials and especially the skin, the lips and/or the integuments, and which additionally can have an appearance, a feel, a smell and optionally a flavor that is pleasing to the user.

Examples of organic solvents that may be mentioned include C1-C4 lower alcohols, such as ethanol and isopropanol; polyols and polyol ethers, for instance 2-butoxyethanol, propylene glycol, propylene glycol monomethyl ether and diethylene glycol monomethyl ether and diethylene glycol monoethyl ether, and also aromatic alcohols, for instance benzyl alcohol or phenoxyethanol, and mixtures thereof.

The solvents are preferably present in proportions preferably of between 1% and 40% by weight approximately and even more preferably between 5% and 30% by weight approximately relative to the total weight of the final (cosmetic treatment) composition.

At least one additive conventionally used in cosmetics may also be added to the (cosmetic treatment) compositions obtained according to the process of the present invention. Examples of such additives that may be mentioned include anionic, cationic, nonionic, amphoteric or zwitterionic surfactants, or mixtures thereof; anionic, cationic, nonionic, amphoteric or zwitterionic polymers, or mixtures thereof; mineral or organic thickeners, and in particular anionic, cationic, nonionic and amphoteric polymeric associative thickeners; antioxidants; penetrating agents; sequestering agents; fragrances; buffers; dispersants; conditioning agents other than those described above, for instance silicone oils; film-forming agents; preserving agents and opacifiers.

The above additives are generally present in an amount for each of them of between 0.01% and 20% by weight relative to the weight of the final composition.

Needless to say, a person skilled in the art will take care to select this or these optional complementary compounds such that the advantageous properties intrinsically associated with the (cosmetic treatment) composition in accordance with the invention are not, or are not substantially, adversely affected by the addition(s) envisaged.

The pH of the final (cosmetic treatment) composition is generally between 3 and 12 and preferably between 5 and 11. It may be adjusted to the desired value using acidifying or basifying agents usually used in cosmetics, or alternatively using standard buffer systems.

Among the acidifying agents that may be mentioned, for example, are mineral or organic acids such as hydrochloric acid, orthophosphoric acid, sulfuric acid, carboxylic acids such as acetic acid, tartaric acid, citric acid and lactic acid, and sulfonic acids.

Among the basifying agents that may be mentioned, for example, are ammonia, alkaline carbonates, alkanolamines such as mono-, di- and triethanolamine and derivatives thereof, sodium hydroxide, potassium hydroxide and the compounds of the following formula:

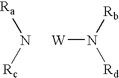

in which W is a propylene residue that is optionally substituted with a hydroxyl group or a C1-C4 alkyl radical; Ra, Rb, Rc and Rd, which may be identical or different, represent a hydrogen atom, a C1-C4 alkyl radical or a C1-C4 hydroxyalkyl radical.

The final (cosmetic treatment) composition may be in various forms, such as in the form of liquids, creams or gels, or in any other form that is suitable for treating keratin materials, and especially the skin or the hair, and more particularly the hair.

The final (cosmetic treatment) composition may be used, for example, as a shampoo, a rinse-out or leave-in conditioner, a deep-down care mask, a shower gel, or a lotion or cream for treating keratin materials.

The present invention also relates to a (cosmetic) process for treating keratin materials, comprising the preparation of a (cosmetic treatment) composition according to the process as defined above, and its application to the keratin materials, for example by means of an operator or by means of a device not requiring any human intervention. The application time may range between 15 seconds and one hour.

Before application, the (cosmetic treatment) composition obtained according to the process of the invention may be mixed with a cosmetically acceptable medium and/or with one or more additives conventionally used in cosmetics, as described above.

Another embodiment consists in preparing at least two (cosmetic treatment) compositions according to the process of the invention, mixing them together, and optionally adding a cosmetically acceptable medium and/or one or more additives conventionally used in cosmetics, as described above, and then in applying the final composition obtained to keratin materials.

The examples below further illustrate the present invention, but do not limit it.

### EXAMPLES

### Example 1

The solid ingredients below are mixed together in the proportions indicated as weight percentages relative to the total weight of solid mixture:

| | |
|---|---|
| Taurine (2-aminoethylsulfonic acid) | 20% |
| Bis(2-ethylhexyl)sulfosuccinate in the form of the | 40% |
| monosodium salt sold under the trade name Aerosol OT 100% by | |
| the company Cytec | |
| Hydroxypropyl guar sold under the trade name Jaguar HP 8 | 40% |
| by the company Rhodia | |

5 g of this mixture are placed in a commercial espresso machine. Steam is then passed through until a composition (A) having a final volume of 50 ml is obtained.

A (cosmetic treatment) composition ready to be applied to the hair is thus obtained. Hair treated with this composition has more body.

Two parts by weight of composition (A) can be added to one part by weight of an aqueous composition (B) containing 1% by weight of hydroxyethylcellulose to facilitate the application.

### Example 2

The solid ingredients below are mixed together in the proportions indicated as weight percentages relative to the total weight of solid mixture:

| | |
|---|---|
| L-Glycine | 20% |
| Powdered sodium lauryl sulfate sold under the trade name | 40% |
| Texapon ® Z 95 P by the company Cognis | |
| Crosslinked C10-C30 alkyl methacrylate/acrylic acid | 40% |
| copolymer sold under the trade name Carbopol ETD 2020 by the | |
| company Noveon | |

5 g of this mixture are placed in a commercial espresso machine. Steam is then passed through until a composition (A) having a final volume of 50 ml is obtained.

A (cosmetic treatment) composition ready to be applied to the hair is thus obtained. Hair treated with this composition has more body.

Two parts by weight of composition (A) can be added to one part by weight of an aqueous composition (B) containing 1% by weight of hydroxyethylcellulose to facilitate the application.

The above written description of the invention provides a manner and process of making and using it such that any person skilled in this art is enabled to make and use the same, this enablement being provided in particular for the subject matter of the appended claims, which make up a part of the original description and including a process for preparing a (cosmetic) composition useful for treating keratin materials, wherein it comprises a step of percolating fluid comprising at least steam, at a pressure of at least 3 bar through at least one amino acid, oligopeptide, peptide, and/or optionally modified, optionally hydrolysed protein, in solid or pasty form.

As used herein, the phrases “selected from the group consisting of,” “chosen from,” “selected from,” and the like include mixtures of the specified materials. In this regard, the term “at least one of an amino acid, oligopeptide, peptide, optionally modified, optionally hydrolysed protein, and mixtures thereof, in solid or pasty form” includes mixtures of two amino acids, mixtures of an amino acid with a peptide, mixtures of three peptides and one oligopeptide, etc.

Where compounds are described as, e.g., “cellulose and its derivatives” an alternate is “cellulose and cellulose compounds” where cellulose derivatives and compounds share a common core/structure with cellulose. One of ordinary skill in the art knows how to identify derivatives and compounds of compound X based on the structure of X and the similarity in structure of the derivatives and compounds.

Where a term is enclosed by parentheses it is an optional modifier. For example, the term “(cosmetic treatment) composition” describes both a composition in general and a cosmetic treatment composition.

All references, patents, applications, tests, standards, documents, publications, brochures, texts, articles, etc. mentioned herein are incorporated herein by reference. Where a numerical limit or range is stated, the endpoints are included. Also, all values and subranges within a numerical limit or range are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein.

1. A process for preparing a composition, comprising percolating a fluid comprising steam, at a pressure of at least 3 bar, through at least one of an amino acid, oligopeptide, peptide, optionally modified, optionally hydrolysed protein, and mixtures thereof, in solid or pasty form.

2. The process according to claim 1, wherein the fluid comprises steam and at least one of liquid water, one or more cosmetically acceptable liquid and/or gaseous solvents, and mixtures thereof.

3. The process according to claim 1, wherein the amino acid is natural.

4. The process according to claim 1, wherein the acid function(s) of the amino acid is (are) carboxylic, sulfonic, phosphonic or phosphoric.

5. The process according to claim 1, comprising percolating a fluid comprising steam, at a pressure of at least 3 bar, through at least one amino acid wherein the amino acid is chosen from aspartic acid, glutamic acid, alanine, arginine, asparagine, carnitine, cysteine, glutamine, glycine, histidine, isoleucine, leucine, methionine, N-phenylalanine, proline, serine, taurine, threonine, tryptophan, tyrosine, valine, polylysine, polyarginine, polyaspartic acid, and mixtures thereof.

6. The process according to claim 5, wherein the amino acid is chosen from arginine, cysteine, glycine, taurine and mixtures thereof.

7. The process according to claim 1, wherein the oligopeptide, the peptide and the hydrolysed or non-hydrolysed, modified or unmodified protein are chosen from TYR-GLY-GLY-PHE-LEU, H-LYS-THR-THR-DAP-SER-OH triacetate, HIS-SER-GLN-GLY-THR-PHE, palmitoyl-TYR-GLY-GLY-PHE-LEU and palmitoyl-GLY-GLN-PRO-ARG; plant proteins derived from plants in native form or present in flours; animal proteins produced by species of the animal kingdom; protein hydrolysates obtained by chemical or enzymatic hydrolysis; chemically modified proteins or protein hydrolysates, and mixtures thereof.

8. The process according to claim 7, wherein the oligopepetide, the peptide and the protein are chosen from soybean proteins, oat proteins, pea proteins, apricot or kernel proteins, wheat gliadin, Triticum aestivum wheat proteins, extensin, thaumatin; wheatgerm flour, oat flour, soybean flour, white lupin flour, wholewheat flour, rice flour, camellia flour, barley flour, rye flour, chestnut flour, quinoa flour, cornflour; whey proteins, casein, silk proteins, transglutin, lactalbumins, lactoglobulins, lactoferrins, egg white proteins including albumin, keratins including those derived from feathers, collagen and gelatin; peptides or protein hydrolysates from soybean, wheat, corn, potato or pea; ethyl or propyl esters of wheat peptides, α-lactalbumbin palmitoyl stearoyl, quaternized protein hydrolysates, and mixtures thereof.

9. The process according to claim 1, wherein the at least one amino acid, the oligopeptide, the peptide and the protein in solid or pasty form are present as a mixture with at least one adjuvant.

10. The process according to claim 9, wherein the adjuvant is chosen from clays, salts, anionic, nonionic, cationic or zwitterionic surfactants, natural or synthetic thickeners, optionally modified starch, glass beads, silica, Nylon, alumina, titanium dioxide, zeolites, polymethyl methacrylate (PMMA), chitosan, maltodextrin, cyclodextrin, mono- or disaccharides, zinc oxide, zirconium oxide, silica beads, talc, borosilicates, especially calcium borosilicate, polyethylene, polytetrafluoroethylene (PTFE), cellulose and its derivatives, superabsorbent compounds, magnesium carbonate, calcium carbonate, polyacrylamide, porous hydroxyapatite, sawdust, wrack powder, crosslinked polyvinylpyrrolidone, calcium alginate, active charcoal, poly(vinylidene chloride/acrylonitrile) particles, and mixtures thereof.

11. The process according to claim 9, wherein the at least one amino acid, oligopeptide, peptide and/or protein are present in an amount of from 0.5% to 99% by weight, relative to the total weight of amino acid(s), oligopeptide(s), peptide(s) and/or protein(s) and adjuvant(s) in solid or pasty form.

12. The process according to claim 9, wherein the composition obtained comprises, in addition to the amino acid(s), oligopeptide(s), peptide(s) and/or protein(s) and the component(s) of the fluid, all or some of the adjuvant(s) present in the mixture in solid or pasty form.

13. The process according to claim 1, wherein the percolation step is performed with a fluid at a pressure ranging from 3 to 30 bar.

14. The process according to claim 13, wherein the percolation step is performed with a fluid at a pressure of at least 10 bar.

15. A composition prepared by a process comprising percolating a fluid comprising steam, at a pressure of at least 3 bar, through at least one of an amino acid, oligopeptide, peptide, optionally modified, optionally hydrolysed protein, and mixtures thereof, in solid or pasty form.

16. The composition according to claim 15, wherein said composition does not comprise any preserving agent.

17. A process, comprising applying the composition of claim 15 to keratin material.

18. The process according to claim 17, wherein the composition is applied to the keratin material by a device not requiring any human intervention.

19. A process according to claim 17, wherein, before application, the composition is mixed with a cosmetically acceptable medium and/or with one or more additives used in cosmetics and or with another composition prepared by a process comprising percolating a fluid comprising steam, at a pressure of at least 3 bar, through at least one of an amino acid, oligopeptide, peptide, optionally modified, optionally hydrolysed protein, and mixtures thereof, in solid or pasty form.

20. A device for packaging a composition, comprising a closed housing delimited by at least one wall that is at least partially permeable to steam at a pressure of at least 3 bar, the device having therein a composition comprising at least one of an amino acid, oligopeptide, peptide, optionally modified, optionally hydrolysed protein, and mixtures thereof, in solid or pasty form, the device being optionally delimited by two sealed sheets or by a tray closed with a lid.
